(19) United States
(12) Patent Application Publication
(10) Pub. No.: US 2017/0242150 A1
(43) Pub. Date: Aug. 24, 2017

(54) MID-INFRARED CEMENT SENSOR

(71) Applicant: SCHLUMBERGER TECHNOLOGY CORPORATION, Sugar Land, TX (US)

(72) Inventors: Timothy Jones, Cambridge (GB); Nathan Lawrence, Cambridge (GB); Go Fujisawa, Sagamihara (JP)

(21) Appl. No.: 15/511,343

(22) PCT Filed: Sep. 11, 2015
(86) PCT No.: PCT/US2015/049554

§ 371 Date: Mar. 15, 2017

(30) Foreign Application Priority Data
Sep. 15, 2014 (GB) 1416260.6

(51) Int. Cl. G01V 8/10; E21B 47/00

(52) U.S. Cl. CPC G01V 8/10; E21B 47/0005; G01N 33/383

(57) ABSTRACT

A sensor is provided for monitoring cement. An internal reflection is contacted with the cement and a mid-infrared light source directs a beam of mid-infrared radiation into said window for attenuated internal reflection at an interface between the window and the cement. The reflected infrared radiation is passed through a first narrow bandpass filter that preferentially transmits mid-infrared radiation over a band of wavelengths corresponding to an absorbance peak of a species associated with the cement to filter internally reflected mid-infrared radiation received from the window. An infrared detector detects filtered mid-infrared radiation transmitted through the first filter and a processor measures the intensity of the detected mid-infrared radiation transmitted through the first filter, and determines therefrom an amount of the species associated with the cement.

### BACKGROUND

Embodiments of the present disclosure relate to mid-infrared sensor for monitoring cement.

The analysis of chemical composition of fluid samples from hydrocarbon wells for the determination of phase behaviour and chemical composition is a critical step in the monitoring and management of a hydrocarbon well as well as the evaluation of the producibility and economic value of the hydrocarbon reserves. Similarly, the monitoring of fluid composition during production or other operations can have an important bearing on reservoir management decisions. Similarly, determination of phase behaviour and chemical composition is important in pipelines and the like used to convey/transport hydrocarbons from the wellhead, including subsea pipelines.

Several disclosures have described analysis of specific gases in borehole fluids in the downhole environment using near-infrared (e.g. λ=1-2.5 μm) spectral measurements. For example, U.S. Pat. No. 5,859,430 describes the use of near-infrared spectroscopy to determine quantitatively the presence of methane, ethane and other simple hydrocarbons in the gas phase. The gases were detected using the absorption of near-infrared radiation by the overtone/combination vibrational modes of the molecules in the spectral region 1.64-1.75 μm.

More recently, U.S. Pat. No. 6,995,360 describes the use of mid-infrared radiation with a wavelength k=3-5 μm to monitor gases in downhole environments, and U.S. Patent Publication No. 2012/0290208 proposes the use of mid-infrared radiation to monitor sequestered carbon dioxide dissolved into the liquid solutions of saline aquifers.

There are however many technical problems with using mid-infrared sensors in the hydrocarbon industry and processing information from such sensors. Additionally, the utility of mid-infrared spectroscopy for in-situ cement monitoring has not previously been recognized.

### SUMMARY

Embodiments of the present disclosure for monitoring cement structures downhole. Such structures can be important in the petrochemical industry for, among other things, well safety and productivity. Merely by way of example, embodiments of the present disclosure provide a system and a method, for among other things monitoring casing cement integrity.

Embodiments of the present disclosure are at least partly based on the recognition that such monitoring can be achieved by monitoring species associated with cement using a sensor based on mid-infrared radiation absorbance.

Accordingly, in a first aspect, embodiments of the present disclosure provide a sensor for monitoring cement, the sensor comprising:
an internal reflection window configured in use to contact with the cement;
a mid-infrared light source configured to direct a beam of mid-infrared radiation onto said window to produce an internally reflected beam of mid-infrared radiation from an interface between the window and the cement;
a first narrow bandpass filter configured to filter the internally reflected beam of mid-infrared radiation received from the window, wherein the first narrow bandpass filter preferentially transmits mid-infrared radiation over a band of wavelengths corresponding to an absorbance peak of a species associated with the cement;
an infrared detector configured to detect filtered mid-infrared radiation transmitted through the first narrow bandpass filter; and
a processor arrangement, operably coupled to the infrared detector and configured to measure an intensity of the detected mid-infrared radiation transmitted through the first narrow bandpass filter and determine an amount (e.g. a concentration) of the species from the measured intensity.

In an embodiment of the present disclosure, by following changes in the amount of the species, properties of the cement such as extent of setting, degradation, fluid flux etc. may be determined.

As discussed below, the sensor may be part of a sensor arrangement e.g. with a further similar sensor for obtaining a reference intensity or the like.

In a second aspect, embodiments of the present disclosure provide for the use of the sensor, or sensor arrangement, of the first aspect to monitor cement. For example, in one embodiment, a method of monitoring cement may comprise: deploying the sensor of the first aspect such that the internal reflection window is in direct contact with the cement; and operating the sensor to determine an amount of a species associated with the cement. In some embodiments of the present disclosure, the deployment of the sensor may be downhole. In some embodiments of the present disclosure, the deployment of the sensor may include embedding the sensor in a cement structure, such as cement of a wellbore casing.

In a third aspect, embodiments of the present disclosure provide a well tool including the sensor, or sensor arrangement, of the first aspect. The well tool can be configured to deploy the sensor at a location for embedding in a downhole cement structure, such as the cement of a wellbore casing.

Optional features of embodiments of the present disclosure will now be set out. These are applicable singly or in any combination with any aspect of an embodiment of the present disclosure.

By “mid-infrared radiation,” it is meant that in embodiments of the present disclosure the radiation has a wavelength in the range from about 2 to 20 μm, and in some embodiments from about 3 to 12 μm or from about 3 to 10 μm.

In an embodiment of the present disclosure, the first narrow bandpass filter may be configured such that its wavelength transmission band is substantially temperature invariant over all temperatures in the range from −25 to 25° C. Temperatures in subsea environments can vary, e.g. from about −25 to 25° C. By using such a temperature invariant filter, the sensitivity of the sensor to shifts in temperature of its surroundings can be greatly reduced, improving the accuracy with which the amount of the cement species is determined.

By “substantially temperature invariant,” it is meant herein that the variance in embodiments of the present disclosure is at most about 0.1 nm/° C. In some embodiments, the variance is at most about 0.05, 0.03, 0.02 or 0.01 nm/° C.

In embodiments of the present disclosure, each filter may comprise an interference filter. Merely by way of example, in some embodiments of the present disclosure, each filter may include a substrate, formed of Si, SiO₂, Al₂O₃, Ge or ZnSe and/or the like, and at each opposing side of the substrate alternating high and low refractive index layers may be formed. In some embodiments of the present disclosure, the high refractive index layers can be formed of PbTe, PbSe or PbS and the low refractive index layers can be formed of ZnS, ZnSe and/or the like.

In embodiments of the present disclosure, each filter may have three or more half wavelength cavities. Many conventional filters display unacceptably high band shifts with increasing temperature. For example, shifts in the range of 0.2 to 0.6 nm/° C. are typical in prior filters. Transmissivities also tend to reduce with increasing temperature. These properties, have prevented/limited development of mid-infrared sensors, especially in the petrochemical industry.

However, in accordance with embodiments of the present disclosure, by using a PbTe-based, a PbSe-based, a PbS-based interference filter and/or the like it is possible to substantially reduce band shifts and transmissivity reductions. For example, a PbTe-based interference filter, in accordance with an embodiment of the present disclosure, may have a band shift of only about 0.03 nm/° C. or less. As an alternative to PbTe, PbSe, PbS or the like, the high refractive index layers can be formed, in some embodiments of the present disclosure, of Ge or the like.

In embodiments of the present disclosure, a reference intensity may be used in the determination of the amount of the species in the fluid. Thus a sensor arrangement may include the sensor of the first aspect and a further similar sensor which can be used to obtain this reference intensity. The further sensor can have the same features as the first sensor except that its narrow bandpass filter transmits mid-infrared radiation over a band of wavelengths corresponding to a reference portion of the absorbance spectrum of the cement. In such a scenario, the processor arrangement can be a shared processor arrangement of both sensors.

Another option, however, is to obtain the reference intensity using the first sensor. For example, the sensor may further include a second narrow bandpass filter transmitting mid-infrared radiation over a band of wavelengths corresponding to a reference portion of the absorbance spectrum of the cement, the or a further infrared detector detecting filtered mid-infrared radiation transmitted through the second filter, and the processor arrangement measuring the reference intensity of the detected mid-infrared radiation transmitted through the second filter and using the measured reference intensity in the determination of the amount of the species associated with the cement. The first and second filters may be selectably positionable between a single detector and the window, or each of the first and second filters can have a respective detector. The second narrow bandpass filter may be configured such that its wavelength transmission band is substantially temperature invariant over all temperatures in the range from about 25 to 150° C. Optional features of the first narrow bandpass filter pertain also to the second narrow bandpass filter.

The sensor may be able to measure the amounts of more than one species associated with the cement. For example, the sensor may include a plurality of the first narrow bandpass filters, each transmitting mid-infrared radiation over a band of wavelengths corresponding to an absorbance peak of a respective species associated with the cement, the or a respective further infrared detector detecting the filtered mid-infrared radiation transmitted through each first filter, and the processor arrangement measuring the intensity of the detected mid-infrared radiation transmitted through each first filter and determining therefrom an amount of each species associated with the cement. The first filters may be selectably positionable between a single detector and the window, or each first filter can have a respective detector.

When the sensor is able to measure monitor more than one species, the determined amounts of the species associated with the cement can be in the form of a ratio of the concentrations of the species.

In embodiments of the present disclosure, the transmission band(s) of the first filter(s) may correspond to absorbance peaks of, for example, water, oil, CO₂, (CaO)₃SiO₂, 3CaO2SiO₂.4H₂O (known as C—S—H gel), and/or carbonate. In embodiments of the present disclosure, cement setting can be associated with a decrease in (CaO)₃SiO₂ peaks, and an increase in C—S—H peaks. Cement debonding can be associated with a decrease in C—S—H peaks and an increase in water and/or oil peaks. The presence of CO₂ can be associated with an increase in CO₂ peaks and an increase in carbonate peaks. The first narrow bandpass filter can preferentially transmit mid-infrared radiation over a band of wavelengths corresponding to a 943 cm⁻¹ absorbance peak for C—S—H gel, a 925 cm⁻¹ or an 890 cm⁻¹ absorbance peak for (CaO)₃.SiO₂, a 1430 cm⁻¹ absorbance peak for carbonate, a 3330 cm⁻¹ absorbance peak for water, a 2900 cm⁻¹ absorbance peak for oil, or a 2350 cm⁻¹ absorbance peak for CO₂.

In some embodiments of the present disclosure, the beam of mid-infrared light may be pulsed. This can be achieved, for example, by providing a mechanical chopper between the source and the window, or by pulsing the source.

In some embodiments of the present disclosure, the source may be a broad band thermal source or a narrower band source such as a light emitting diode or a laser.

In some embodiments of the present disclosure, the detector may be a thermopile, a pyroelectric or (particularly in subsea applications, where the low ambient temperatures can provide cooling) a photodiode detector.

In some embodiments of the present disclosure, the window may comoprise a diamond window, a sapphire window or the like. In some embodiments of the present disclosure, diamond windows can be formed by chemical vapour deposition. Sapphire has a cut off for mid-infrared radiation at wavelengths of about 5 to 6 microns, but sapphire windows can generally be formed more cheaply than diamond windows. Thus, for absorption peaks below the cut off (such as the CO₂ absorption peak at about 4.3 microns), sapphire can be a useful alternative to diamond.

In some embodiments of the present disclosure, the sensor may be located downhole, for example embedded in a cement structure such as the cement of a wellbore casing.

In an embodiment of the present disclosure, the monitored species may be:
a compound forming the cement, or one or more compounds in a mixture of compounds forming the cement,
a constituent group of a compound forming the cement, or a constituent group common to one or more compounds in a mixture of compounds forming the cement,
one or more compounds or ions dissolved in or forming a fluid, or
a constituent group of a compound or ion dissolved in or forming a fluid, or a constituent group common to one or more compounds or ions dissolved in or forming a fluid.

### BRIEF DESCRIPTION OF THE DRAWINGS

Embodiments of the present disclosure will now be described by way of example with reference to the accompanying drawings in which:

FIG. 1 shows schematically, in accordance with embodiments of the present invention, (a) a mid-infrared sensor, and (b) the sensor implemented as a module in a toolstring;

FIG. 2 shows schematically a narrow bandpass filter, in accordance with an embodiment of the present disclosure, based on Fabry-Perot interferometry;

FIG. 3 shows variation of dλₘ/λₘdT for a suite of filters fabricated with ZnSe as the low refractive index material and PbTe as the high refractive index material;

FIG. 4 shows plots of transmissivity against wavelength at a range of temperatures from 25 to 200° C. for (a) a PbTe-based filter having a pass band centred at 4.26 μm, and (b) a PbTe-based filter having a pass band centred at 12.1 μm;

FIG. 5 shows (a) a reference intensity spectrum Iₒ obtained from a fluid not containing a given species, (b) an intensity spectrum I obtained from the fluid containing the species, and (c) the absorbance spectrum of the species;

FIG. 6 shows intensity spectra obtained for dodecane dissolved in deuterated chloroform for increasing concentrations of dodecane, the spectra being superimposed with transmissivity plots for a first filter having a pass band of 3000 to 2800 cm⁻¹, and a second filter having a pass band of 2000 to 1800 cm⁻¹;

FIG. 7 shows a plot of modified absorbance A′ against hydrocarbon content for dodecane dissolved in deuterated chloroform;

FIG. 8 shows a plot of absorbance against dissolved CO₂ concentration in water or hydrocarbon;

FIG. 9 shows schematically a cased wellbore with embedded cement integrity sensors, in accordance with embodiments of the present invention;

FIG. 10 shows a time evolution of a mid-infrared absorbance spectrum for setting cement at 25° C.;

FIG. 11 shows a time evolution of a mid-infrared absorbance spectrum for setting cement at 200° C.; and

FIG. 12 shows a time evolution of a mid-infrared absorbance spectrum for a plurality of species associated with setting cement of a wellbore casing.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiment(s) only, and is not intended to limit the scope, applicability or configuration of the invention. Rather, the ensuing description of the preferred exemplary embodiment(s) will provide those skilled in the art with an enabling description for implementing a preferred exemplary embodiment of the invention, it being understood that various changes may be made in the function and arrangement of elements without departing from the scope of the invention.

Specific details are given in the following description to provide a thorough understanding of the embodiments. However, it will be understood by one of ordinary skill in the art that embodiments maybe practiced without these specific details. For example, well-known circuits, processes, algorithms, structures, and techniques may be shown without unnecessary detail in order to avoid obscuring the embodiments.

Also, it is noted that the embodiments may be described as a process which is depicted as a flowchart, a flow diagram, a data flow diagram, a structure diagram, or a block diagram.

Although a flowchart may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine readable mediums for storing information. The term “computer-readable medium” includes, but is not limited to portable or fixed storage devices, optical storage devices, wireless channels and various other mediums capable of storing, containing or carrying instruction(s) and/or data.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine readable medium such as storage medium. A processor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

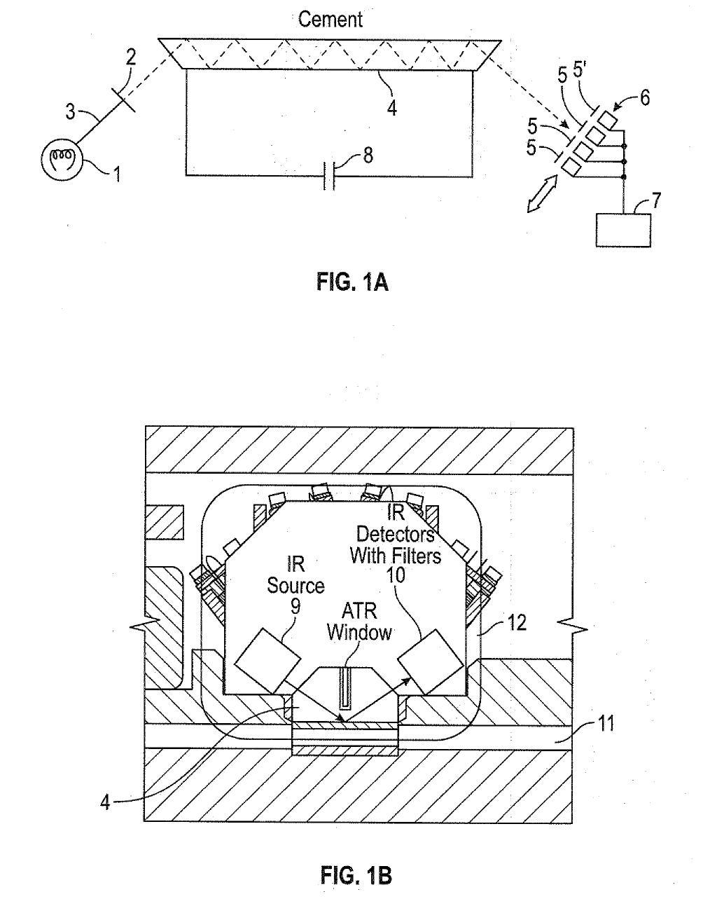

FIG. 1(a) shows schematically a mid-infrared sensor, in accordance with an embodiments of the present disclosure, having a thermal broad band mid-infrared source 1, a mechanical chopper 2 that pulses a beam 3 of mid-infrared radiation which issues from the source, a diamond window 4, a set of selectively movable first narrow bandpass filters 5 and a second narrow bandpass filter 5′, respective mid-infrared detectors 6 for the filters, and a processor arrangement 7. The sensor is encased in a protective housing which allows the sensor to be deployed downhole, the window 4 being positioned for contact with the cement to be monitored. Mid-infrared waveguides (not shown) optically connect the source, window and the detectors. Suitable waveguides can be formed from optical fibres (e.g. hollow fibres or chalcogenide fibres), solid light pipes (e.g. sapphire pipes), or hollow light pipes (e.g. air or vacuum filled) with a reflective (e.g. gold) coating.

As the detector 6 changes its output with its temperature, even small changes in temperature can cause a large drift in signal output. However, pulsing the beam 3 allows the output signal of the detector to be frequency modulated, enabling removal of the environmental temperature effects from the signal. More particularly, the environment effects can be largely removed electronically by a high pass filter, because the time constant for environment effects tends to be much longer than the signal frequency. In embodiments of the present disclosure, the detector output may be AC-coupled to an amplifier. The desired signal can then be extracted e.g. electronically by lock-in amplification or computationally by Fourier transformation.

Instead of the thermal source 1 and the mechanical chopper 2, the pulsed beam 3 may be produced e.g. by a pulsable thermal source, light emitting diode or laser source. Pulsing the source in this way can give the same benefit of frequency modulation measurement, plus it can reduce resistive heating effects.

The beam 3 enters at one edge of the window 4, and undergoes a number of total internal reflections before emerging from the opposite edge. The total internal reflection of the infrared radiation at the liquid side of the window is accompanied by the propagation of an evanescent wave into the cement. As the liquid preferentially absorbs certain wavelengths, depending on its chemical composition, this causes the emerging beam to have a characteristic variation in intensity with wavelength.

The beam 3 enters at one edge of the window 4, and undergoes a number of total internal reflections before emerging from the opposite edge. The total internal reflection of the infrared radiation at the cement side of the window is accompanied by the propagation of an evanescent wave into the cement. As the cement preferentially absorbs certain wavelengths, depending on its chemical composition, this causes the emerging beam to have a characteristic variation in intensity with wavelength.

The window 4 is mechanically able to withstand the high pressures and temperatures typically encountered downhole. It is chemically stable to fluids encountered downhole and is transparent in the mid-IR wavelength region. Candidate materials for the window are diamond and sapphire.

The first narrow bandpass filters 5 each transmit mid-infrared radiation over a band of wavelengths corresponding to an absorbance peak of a respective species associated with the cement, while the second narrow bandpass filter 5′ transmits mid-infrared radiation over a band of wavelengths corresponding to a reference portion of the absorbance spectrum of the cement. The beam 3 then passes through a selected one of the narrow bandpass filters and is detected at the respective detector 6. Instead of having a plurality of detectors, each movable with its corresponding filter (as indicated by the double-headed arrow), a further option is to have a single detector in front of which the filters are selectively movable.

The detector 6 can be e.g. semiconductor photo-diodes (particularly in subsea applications), thermopiles or pyroelectric detectors.

The processor arrangement 7 receives a signal from the respective detector 6, which it processes to measure the intensity of the detected mid-infrared radiation transmitted through each filter 5, 5′, and, as discussed in more detail below, determines therefrom an amount of the respective species associated with the cement.

FIG. 1(b) shows schematically how the sensor can be implemented as an embedded cement monitor. The source 1 and chopper 2 are contained in a source unit 9 and filters 5, 5′ and detectors 6 are contained in a detector unit 10. These are located close to the window 4 that is in intimate contact with the cement. The sensor is packaged in a protective metal chassis 12 to withstand the high downhole pressure.

### Narrow Bandpass Filters

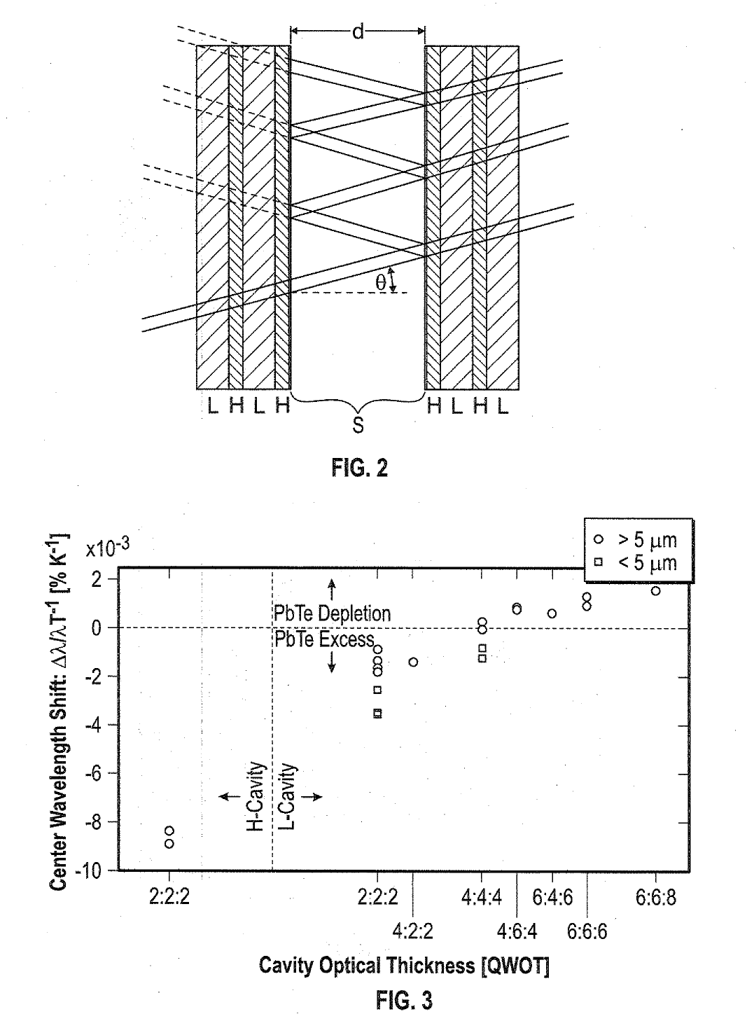

In embodiments of the present disclosure, the narrow bandpass filters 5, 5′ may be based on Fabry-Perot interferometry. As shown in FIG. 2, each filter may have a substrate S of low refractive index and thickness d. On opposing surfaces of the substrate are stacked alternating high-reflectivity dielectric layers of high H and low L refractive index deposited onto the substrate using techniques such as ion-beam sputtering or radical-assisted sputtering. In some embodiments of the present disclosure, each layer in the stacks of alternating layers of high H and low L refractive index has an optical thickness of a quarter wavelength.

The optical thickness nd cos θ of the substrate S, where n is the refractive index of the substrate, is equal to an integer number of half wavelengths λₘ, where λₘ is the peak transmission wavelength, corresponding approximately to the centre wavelength of the pass band of the filter. The condition for the transmission of radiation of wavelength λₘ through the filter is thus mλₘ/2=nd cos θ, where m is an integer.

The spectral region of conventional narrow bandpass dielectric filters designed to operate in the mid-infrared spectral regions shifts systematically to longer wavelengths with increasing temperature. The origin of the change in λₘ with temperature is a change in the material properties with temperature of the dielecric materials that comprise the layers of the filter.

However, an approach described below, in accordance with an embodiment of the present disclosure, provides for the configuration and fabrication of mid-infrared narrow bandpass filters that have substantially temperature invariant optical properties over a wide temperature range.

The approach can be considered by the design of the filter:

(LH)^{x1}(LL)^{y1}(HL)^{x2}(LL)^{y2} . . . (LL)^{yN}(HL)^{xN+1}

consisting of a total of y half wavelength spacers (cavities) LL of low refractive index material in N cycles (y=Σyᵢ), LH being the stacks of xᵢ quarter wavelength layers of alternating of high and low refractive index material in the N cycles. The reflections wavelength of the quarter wavelength reflector stack (which is the only reflection to undergo constructive interference), irrespective of the values of xᵢ and N, can be expressed as:

λₘ=2(n_{L}d_{L}+n_{H}d_{H})

for first order reflections (m=0). The temperature variation of the wavelength in the reflector stack dλₘ/dTβₛ can be expressed as:

$$\frac{d {λ}_{m}}{dT} {\vert}_{s} = 2 {n}_{L} {d}_{L} ( {C}_{L} + \frac{{dn}_{L}}{{n}_{L} dT} ) + 2 {n}_{H} {d}_{H} ( {C}_{H} + \frac{{dn}_{H}}{{n}_{H} dT} )$$

where C_{L} and C_{H} are the coefficients of linear expansion of the low and high refractive index materials, respectively. From eqn.[1] for first order reflection and normal incidence (i.e., m=1 and θ=0°), the corresponding temperature dependence dλₘ/dTβ_{c} of the cavity layer of low refractive index material is given by:

$$\frac{d {λ}_{m}}{dT} {\vert}_{c} = 2 {yn}_{L} {d}_{L} ( {C}_{L} + \frac{{dn}_{L}}{{n}_{L} dT} )$$

noting that y is the total number of half wavelength cavity layers. The total change in wavelength with temperature d□ₘ/dT|_{T} is given by the sum of dλₘ/dT|_{c} and dλₘ/dT|ₛ:

$$\frac{d {λ}_{m}}{dT} {\vert}_{T} = 2 ( 1 + y ) {n}_{L} {d}_{L} ( {C}_{L} + \frac{{dn}_{L}}{{n}_{L} dT} ) + 2 {n}_{H} {d}_{H} ( {C}_{H} + \frac{{dn}_{H}}{{n}_{H} dT} )$$

$$or$$

$$\frac{d {λ}_{m}}{{λ}_{m} dT} {\vert}_{T} = ( 1 + y ) ( {C}_{L} + \frac{{dn}_{L}}{{n}_{L} dT} ) + ( {C}_{H} + \frac{{dn}_{H}}{{n}_{H} dT} )$$

noting that n_{L}d_{L}=n_{H}d_{H} at the temperature for which the filter is designed for use. Clearly dλₘ/dT|_{T} can only be zero if the value of dn/dT for one of the materials is negative. This condition can be fulfilled by high refractive index materials such as PbTe, PbSe or PbS. For close matching of the value of dλₘ/dT|_{T} to zero, the wavelength dependence of nᵢ temperature and wavelength dependence of dnᵢ/dT can be taken into account.

The condition dλₘ/dTβ_{T}=0 is given approximately by:

$$\frac{{dn}_{H}}{{n}_{H} dT} = - ( 1 + y ) \frac{{dn}_{L}}{{n}_{L} dT}$$

noting that Cᵢ is considerably smaller than dnᵢ/nᵢdT for most materials used in mid-infrared filters. The term (1+y) can be chosen to satisfy the above expression depending on the choice of low refractive index material. For example, with ZnSe and PbTe for the low and high refractive index materials, respectively, and using the material values of bulk phases n_{L}=2.43, n_{H}=6.10, dn_{L}/dT=6.3×10⁻⁵ K⁻¹ and dn_{H}/dT=−2.1×10⁻³ K⁻¹ for λₘ=3.4 □m, the expression is satisfied with y=13.3, i.e., approximately 13 half wavelength cavity layers are required to achieve the condition dλₘ/dT|_{T}=0.

There is considerable variation in the values of the material properties (n_{H}, dn_{H}/dT, C_{H}, etc.) that appear in for thin films in a multilayer structure and therefore in the predicted value of dλₘ/λₘdT or the value of y required to achieve the condition dλₘ/λₘdT=0. The uncertainty is particularly severe for the value of dn_{H}/dT for PbTe in view of its magnitude and influence on the value of y. For example, the value of dn/dT for PbTe at λₘ=5 □m has been reported to be −1.5×10⁻³ K⁻¹ by Zemel, J. N., Jensen, J. D. and Schoolar, R. B., “ELECTRICAL AND OPTICAL PROPERTIES OF EPITAXIAL FILMS OF PBS, PBSE, PBTE AND SNTE”, Phys. Rev. 140, A330-A343 (1965), −2.7×10⁻³ K⁻¹ by Piccioli, N., Besson, J. M. and Balkanski, M., “OPTICAL CONSTANTS AND BAND GAP OF PBTE FROM THIN FILM STUDIES BETWEEN 25 AND 300° K.”, J. Phys. Chem. Solids, 35, 971-977 (1974), and −2.8×10⁻³ K⁻¹ by Weiting, F. and Yixun, Y., “TEMPERATURE EFFECTS ON THE REFRACTIVE INDEX OF LEAD TELLURIDE AND ZINC SELENIDE”, Infrared Phys., 30, 371-373 (1990). From the above expression, the corresponding values of y (to the nearest integer) are 9, 17 and 18, respectively.

In view of the uncertainties in the value of dn/dT for PbTe and therefore the number of low refractive index half wavelength spacers required to achieve dλₘ/dT=0, a more useful approach is to determine the experimental value of dλₘ/dT as a function of the optical thickness of the low refractive index cavities for a suite of filters fabricated by the same method. FIG. 3 shows the variation of dλₘ/λₘdT for a suite of filters fabricated with ZnSe as the low refractive index material and PbTe as the high refractive index material. The plot shows that a particular value of dλₘ/λₘdT can be achieved by controlling the ratio of low to high refractive index materials in the filter (i.e., a parameter similar to y in the above expression). FIG. 3 shows that for λₘ<5 μm, the condition dλₘ/λₘdT=0 is met by a 4:4:4 (i.e., 3 full wavelength or 6 half wavelength cavities (y=6)) filter, while for kₘ>5 □m a 6:4:6 (y=8) filter is required.

The approach illustrated by FIG. 3 can be used, in accordance with an embodiment of the present disclosure, to fabricate substantially temperature invariant filters over the entire mid-infrared spectral range. In some embodiments of the present disclosure, the substrate may be formed of Si, SiO₂, Al₂O₃, Ge or ZnSe. In some embodiments of the present disclosure, high refractive index layers can be formed of PbTe, PbSe or PbS, although Ge is also an option. In some embodiments of the present disclosure, the low refractive index layers can be formed of ZnS or ZnSe.

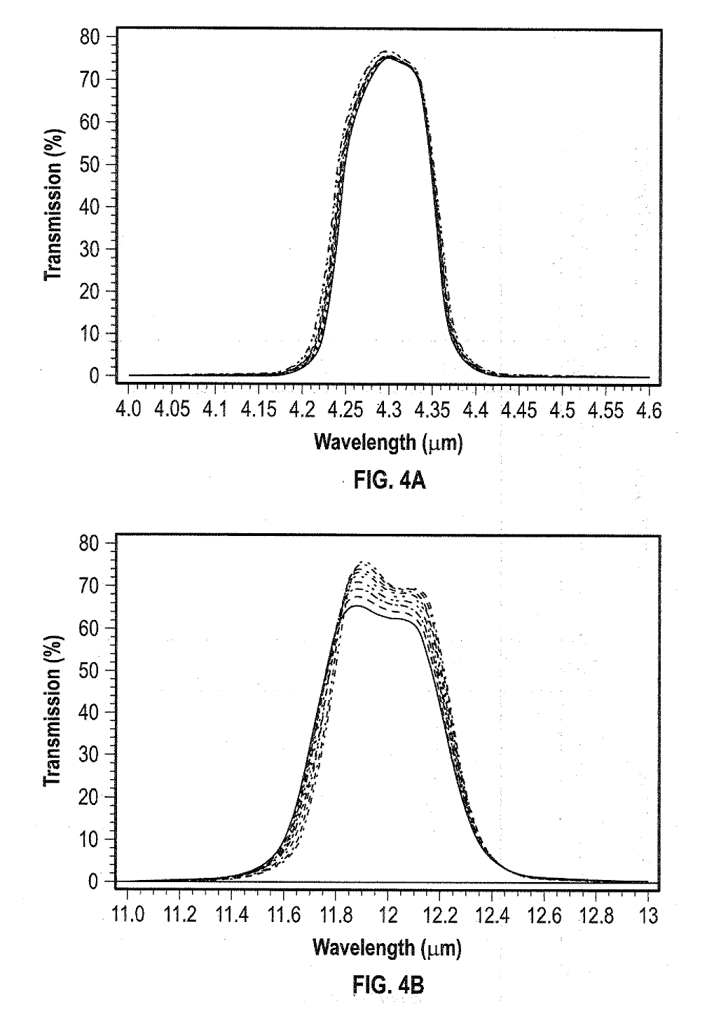

FIG. 4 shows plots of transmissivity against wavelength at a range of temperatures from 25 to 200° C. for (a) a PbTe-based filter having a pass band centred at 4.26 μm with optimum optical matching to the substrate and 3 full wavelength thickness cavities (4:4:4), and (b) a degenerate PbTe-based filter having a pass band centred at 12.1 μm with 3 half wavelength cavities (2:2:2). Similar filters can be produced having pass bands centred at other mid-infrared wavelengths. The value of dλₘ/dT for the λₘ=4.26 □m (4:4:4) filter varies from −0.04 nm/K at 20° C. to +0.03 nm/K at 200° C. and is essentially zero over the temperature range 80-160° C. The value of dλₘ/dT for the λₘ=12.1 □m (2:2:2) filter is −0.21 nm/K, over the temperature range 20-200° C. This allows such filters to deployed downhole or in subsea locations, where temperatures may vary from about 25 to 200° C., without the pass band of the filter shifting to such an extent that it no longer corresponds to the absorbance peak of its respective species. Similarly, filters can be configured for use in subsea locations, where temperatures can vary from about −25 to 25° C., without significant pass band shifting.

Spectroscopy

The Beer-Lambert law applied to the sensor of FIG. 1 provides that:

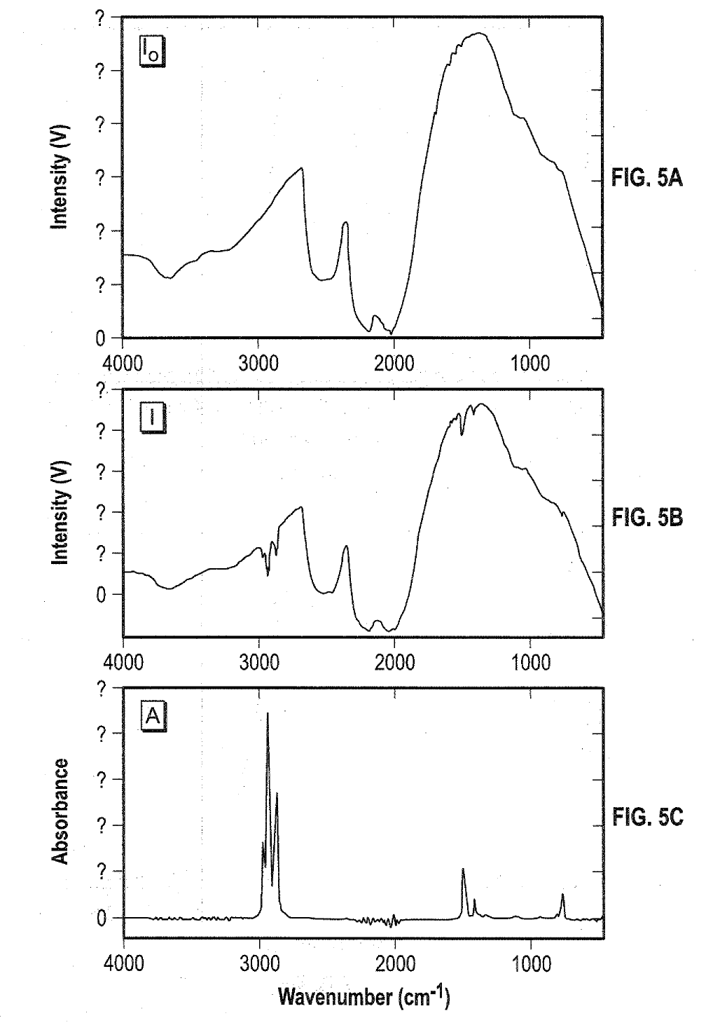

A=−log₁₀(I/I₀)

where A is the absorbance spectrum by a species in a fluid having an absorbance peak at a wavelengths corresponding to the pass band of the filter 5, I is the intensity spectrum of the infrared radiation detected by the detector 6, and I₀ is a reference intensity spectrum. For example, FIG. 5 shows (a) a reference intensity spectrum I₀ obtained from a fluid not containing a given species, (b) an intensity spectrum I obtained from the fluid containing the species, and (c) the absorbance spectrum of the species.

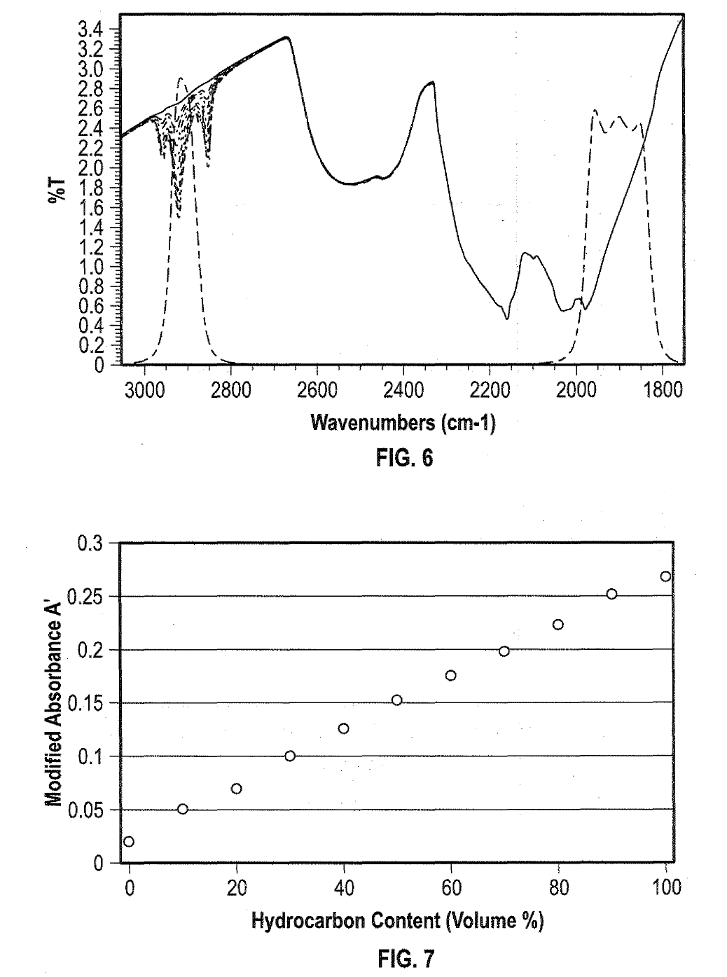

FIG. 6 shows intensity spectra obtained for dodecane dissolved in deuterated chloroform for increasing concentrations of dodecane. With increasing hydrocarbon content there is increased absorption in a first wavenumber range of 3000 to 2800 cm⁻¹. Conversely, the increasing hydrocarbon content has substantially no effect on absorption in a second wavenumber range of 2000 to 1800 cm⁻¹. The second range can thus be used as the reference to the first range. Superimposed on FIG. 6 are transmissivity plots for a first filter having a pass band of 3000 to 2800 cm⁻¹, and a second filter having a pass band of 2000 to 1800 cm⁻¹. Two spectra are thus, in effect, detected by the filters, the first spectrum being the unfiltered spectrum multiplied by the transmissivity of the first filter and the second sub-spectrum being the unfiltered spectrum multiplied by the transmissivity of the second filter. The pass band areas of the spectra (as determined by the strengths of the signals received by the photodiode detectors), correspond to respective intensity measurements BA and BA₀. These are thus used to calculate a modified absorbance A′ for dodecane dissolved in deuterated chloroform which is ln(BA/BA₀).

FIG. 7 shows a plot of modified absorbance A′ against hydrocarbon content for dodecane dissolved in deuterated chloroform. The plot exhibits an approximately linear relationship between A′ and hydrocarbon content.

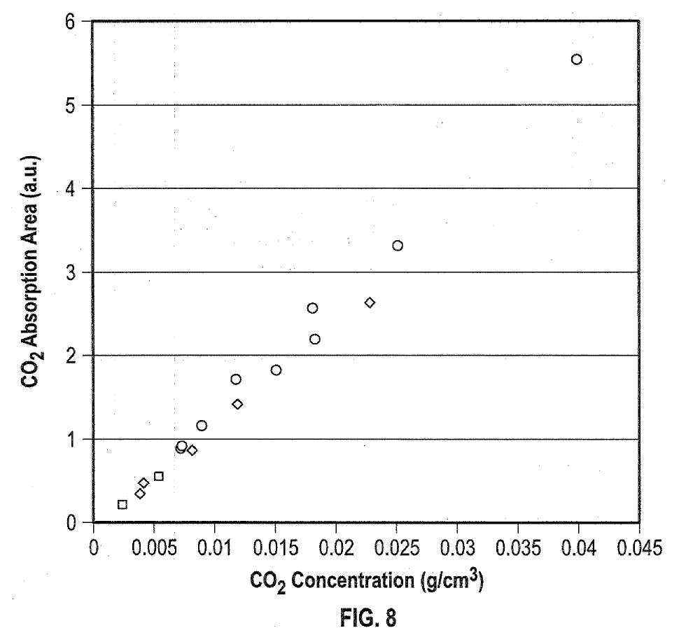

Other species can be monitored in this way. For example, FIG. 8 shows a plot of absorbance against dissolved CO₂ concentration in water or hydrocarbon under the high partial pressures and temperatures typical of oil field wellbore conditions.

Cement Characterisation

During the construction of wellbores, it is common, during and after drilling, to place a tubular body in the wellbore. The tubular body may comprise drillpipe, casing, liner, coiled tubing or combinations thereof. Usually, a plurality of tubular bodies are placed sequentially and concentrically, with each successive tubular body having a smaller diameter than the previous tubular body, set at selected depths as drilling progresses. The purpose of the tubular body is to support the wellbore and to act as a conduit through which desirable fluids from the well may travel and be collected. The tubular body is normally secured in the well by a cement sheath. The cement sheath provides mechanical support and hydraulic isolation between the zones or layers that the well penetrates. he latter function is important because it prevents hydraulic communication between zones that may result in contamination. For example, the cement sheath blocks fluids from oil or gas zones from entering the water table and polluting drinking water. In addition, to optimize a well's production efficiency, it may be desirable to isolate, for example, a gas-producing zone from an oil-producing zone.

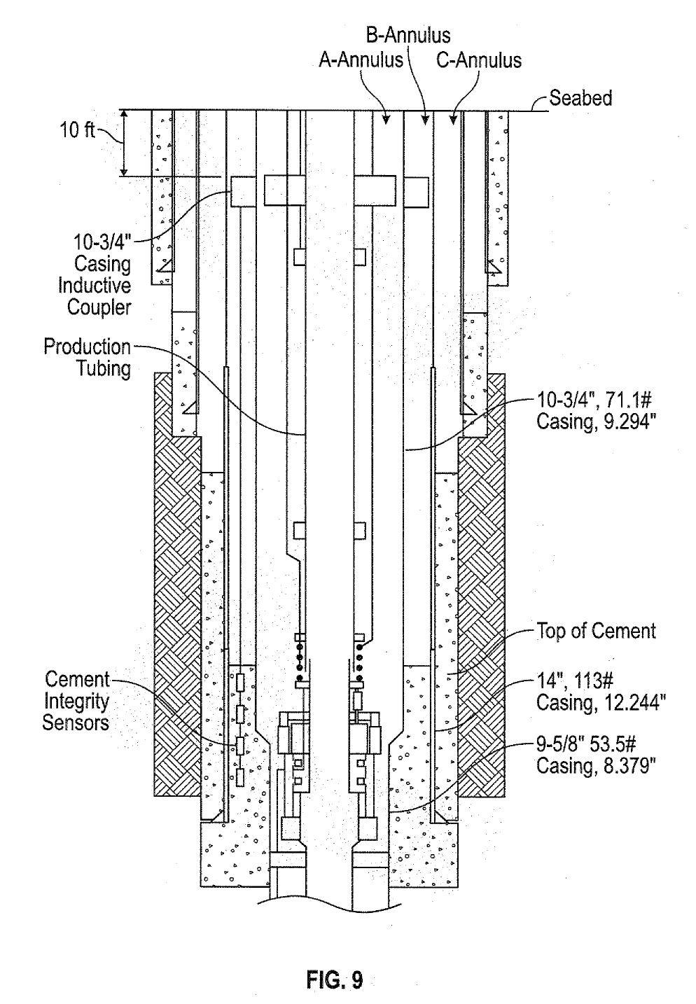

FIG. 9 shows schematically a cased wellbore with a series of coaxial casings, each comprising steel pipe with a respective cement sheath.

The cement sheath achieves hydraulic isolation because of its low permeability. In addition, intimate bonding between the cement sheath and both the tubular body and borehole prevents leaks. However, over time the cement sheath can deteriorate and become permeable. Alternatively, the bonding between the cement sheath and the tubular body or borehole may become compromised. The principal causes of deterioration and debonding include physical stresses associated with tectonic movements, temperature changes and chemical deterioration of the cement.

However, a mid-infrared sensor, of the type discussed above, may be used to characterise downhole cement structures, such as wellbore casings. The ability of the sensor to operate under a full range of downhole temperatures can be particularly advantageous. In FIG. 9 a string of cement integrity sensors is shown embedded in the cement of the lowermost casing such that the window of each sensor is in intimate contact with the surrounding cement.

The sensors can provide real time monitoring of cement setting, cement debonding, and/or CO₂ detection.

The principal phases in cement are: (CaO)₃SiO₂, (CaO)₂.SiO₂, (CaO)₃.Al₂O₃ and (CaO)₄.Al₂O₃.Fe₂O₃. The principal setting reaction is:

2(CaO)₃.SiO₂+7H₂O→3CaO.2SiO₂.4H₂O+3Ca(OH)₂

The reaction product 3CaO.2SiO₂.4H₂O is termed C—S—H gel and its formation causes the cement to set. However, in the presence of added calcium sulphate, the interstitial phase reaction:

(CaO)₃.Al₂O₃+3CaSO₄+32H₂O→(CaO)₃.Al₂O₃.(CaSO₄)₃.32H₂O

can occur. Formation of the reaction product (ettringite) can inhibit the hydration reaction which produces C—S—H gel.

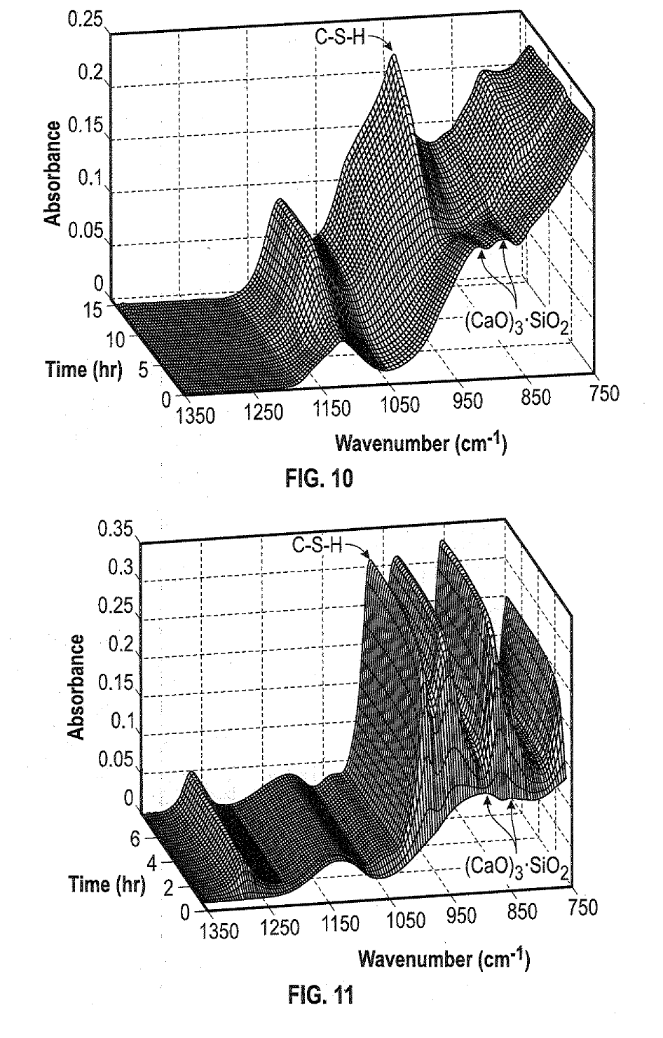

FIG. 10 shows a time evolution of a mid-infrared absorbance spectrum for setting cement at 25° C. Consistent with the principal setting reaction, weak (CaO)₃.SiO₂ peaks at 925 and 890 cm⁻¹ are replaced by an intense peak at 943 cm⁻¹ due to C—S—H gel formation.

FIG. 11 shows another time evolution of a mid-infrared absorbance spectrum for setting cement, but now at 200° C. The different temperature drives the reaction at a different rate and produces different reaction products, but again weak (CaO)₃.SiO₂ peaks at 925 and 890 cm⁻¹ are replaced by an intense peak at 943 cm⁻¹ due to C—S—H gel formation.

Thus by measuring the relative strengths of these peaks, and determining therefrom the relative amounts of the respective phases, the setting reaction can be monitored in real time.

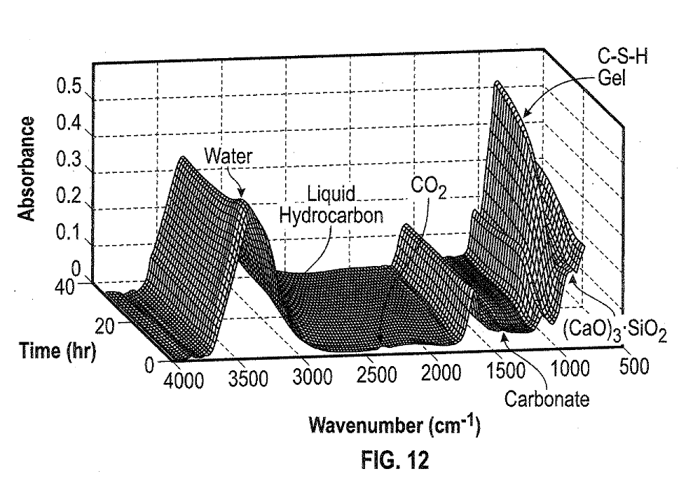

As regards cement debonding, this can lead to the window of a given sensor debonding from the surrounding cement and allowing the ingress of water or oil. Accordingly, debonding can be detected by a reduction in the C—S—H peak at 943 cm⁻¹ and an increase in peaks due to water and liquid hydrocarbons. The respective peaks are indicated in FIG. 12, which shows a time evolution of a mid-infrared absorbance spectrum for a plurality of species associated with setting cement of a wellbore casing.

Also shown in FIG. 12 are a CO₂ peak 2350 cm⁻¹ and a carbonate peak at 1430 cm⁻¹. Ingressing or dissolved CO₂ can react with C—S—H gel and Ca(OH)₂ to form carbonate, which can weaken the cement. However, by measuring the CO₂ and carbonate peaks, the presence or extent of this reaction can be monitored.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the scope of the present disclosure, and that they may make various changes, substitutions and alterations herein without departing from such scope.

All references referred to above are hereby incorporated by reference.

1. A sensor for monitoring cement, the sensor comprising:
an internal reflection window configured in use to contact the cement;
a mid-infrared light source configured to direct a beam of mid-infrared radiation onto the internal reflection window to produce an internally reflected beam of mid-infrared radiation from an interface between the internal reflection window and the cement;
a first narrow bandpass filter configured to filter the internally reflected beam of mid-infrared radiation received from the internal reflection window, wherein the first narrow bandpass filter preferentially transmits mid-infrared radiation over a band of wavelengths corresponding to an absorbance peak of a species associated with the cement;
an infrared detector configured to detect filtered mid-infrared radiation transmitted through the first narrow bandpass filter; and
a processor arrangement, operably coupled to the infrared detector and configured to measure an intensity of the detected mid-infrared radiation transmitted through the first narrow bandpass filter and determine an amount of the species from the measured intensity.

2. The sensor according to claim 1, wherein the first narrow bandpass filter preferentially transmits mid-infrared radiation over at least one of a band of wavelengths corresponding to an absorbance peak of about 943 cm⁻¹ for C—S—H gel; an absorbance peak of about 925 cm⁻¹ or about 890 cm⁻¹ for (CaO)3.SiO2; an absorbance peak of about 1430 cm⁻¹ for carbonate; an absorbance peak of about 3330 cm⁻¹ for water; an absorbance peak of about 2900 cm⁻¹ for oil; or an absorbance peak of about 2350 cm⁻¹ for CO₂.

3. The sensor according to claim 1, wherein the first narrow bandpass filter is configured such that its wavelength transmission band is substantially temperature invariant over all temperatures in the range from about 25° C. to about 150° C.

4. The sensor according to claim 1, further comprising:
a second narrow bandpass filter configured to transmit mid-infrared radiation over a band of wavelengths corresponding to a reference portion of the absorbance spectrum of the cement; and
the or a further infrared detector configured to detect filtered mid-infrared radiation transmitted through the second narrow bandpass filter, wherein:
the processor arrangement is configured to measure a reference intensity of the detected mid-infrared radiation transmitted through the second filter; and
the step of determining an amount of the species from the measured intensity uses the measured reference intensity.

5. The sensor according to claim 1, further comprising:
a plurality of the first narrow bandpass filters each transmitting mid-infrared radiation over a band of wavelengths corresponding to an absorbance peak of a respective species associated with the cement; and
the or a respective further infrared detector detecting the filtered mid-infrared radiation transmitted through each first narrow bandpass filter; wherein
the processor arrangement measures the intensity of the detected mid-infrared radiation transmitted through each of the plurality of the first narrow bandpass filters and the step of determining an amount of the species from the measured intensity comprises determines from the measured intensity through each of the plurality of the first narrow bandpass filters an amount of each species associated with the cement.

6. The sensor according to claim 5, wherein the determined amounts of the species associated with the cement is in the form of a ratio of the concentrations of the species.

7. The sensor according to claim 1, wherein the beam of mid-infrared light is pulsed.

8. The sensor according to claim 1, wherein the internal reflection window is a diamond internal reflection window or a sapphire internal reflection window.

9. The sensor according to claim 1 which is configured for use downhole.

10. Use of the sensor of claim 1 to determine an amount of a species associated with the cement.

11. A method of monitoring cement including:
deploying the sensor of claim 1 such that the internal reflection window is in direct contact with the cement; and
operating the sensor to determine an amount of a species associated with the cement.

12. The method according to claim 11, wherein the deployment of the sensor is downhole.

13. The method according to claim 11, wherein the deployment of the sensor comprises embedding the sensor in cement of a wellbore casing.

14. A well tool including the sensor of claim 1.

15. A method for monitoring cement in a wellbore, the method comprising:
contacting an internal reflection window with the cement;
directing a beam of mid-infrared radiation onto the internal reflection window to produce an internally reflected beam of mid-infrared radiation from an interface between the internal reflection window and the cement;
filtering the internally reflected beam of mid-infrared radiation received from the internal reflection window by passing it through a filter, wherein the filter preferentially transmits mid-infrared radiation over a band of wavelengths corresponding to an absorbance peak of a species associated with the cement;
measuring an intensity of the filtered mid-infrared radiation; and
determining an amount of the species from the measured intensity.

16. The method according to claim 15, wherein the filter preferentially transmits mid-infrared radiation over at least one of a band of wavelengths corresponding to: an absorbance peak of about 943 cm⁻¹ for C—S—H gel; an absorbance peak of about 925 cm⁻¹ or about 890 cm⁻¹ for (CaO)3.SiO2; an absorbance peak of about 1430 cm⁻¹ for carbonate; an absorbance peak of about 3330 cm⁻¹ for water; an absorbance peak of about 2900 cm⁻¹ for oil; or an absorbance peak of about 2350 cm⁻¹ for CO₂.

17. The method according to claim 15, wherein the filter is configured such that its wavelength transmission band is substantially temperature invariant over all temperatures in the range from about 25° C. to about 150° C.

18. The method according to claim 15, further comprising:
passing the internally reflected beam of mid-infrared radiation through a reference filter that transmits mid-infrared radiation over a band of wavelengths corresponding to a reference portion of the absorbance spectrum of the cement;
measuring a reference intensity of the internally reflected beam of mid-infrared radiation passing through the second filter; and
using the reference intensity in the step of determining an amount of the species from the measured intensity.

19. The method according to claim 15, further comprising:
pulsing the beam of mid-infrared light directed onto the internal reflection window.
